SEQUENCE LISTING (13 sequences)

SEQ ID NO: 1 - DNA, 30 bp, Artificial Sequence
  Description of Artificial Sequence Synthetic primer
gagaatcgcttgaacccgggaggtgtaggt

SEQ ID NO: 2 - DNA, 30 bp, Artificial Sequence
  Description of Artificial Sequence Synthetic primer
cctgctgatgcctggcctctttcttctttg

SEQ ID NO: 3 - DNA, 30 bp, Artificial Sequence
  Description of Artificial Sequence Synthetic primer
catcagcccgtctaggaacccagtcatcag

SEQ ID NO: 4 - DNA, 30 bp, Artificial Sequence
  Description of Artificial Sequence Synthetic primer
ctccttcatcccgctacaccacgcatacac

SEQ ID NO: 5 - DNA, 22 bp, Artificial Sequence
  Description of Artificial Sequence Synthetic primer
gcactggcttaggagttggact

SEQ ID NO: 6 - DNA, 21 bp, Artificial Sequence
  Description of Artificial Sequence Synthetic primer
acagacacccaggcctacttg

SEQ ID NO: 7 - DNA, 29 bp, Artificial Sequence
  Description of Artificial Sequence Synthetic primer
gcactggcttaggagttggacttcaaacc

SEQ ID NO: 8 - DNA, 25 bp, Artificial Sequence
  Description of Artificial Sequence Synthetic primer
caactgctgaaagagatgcggtggg

SEQ ID NO: 9 - DNA, 24 bp, Artificial Sequence
  Description of Artificial Sequence Synthetic primer
agcccttggggasttgaattgctg

SEQ ID NO: 10 - DNA, 27 bp, Artificial Sequence
  Description of Artificial Sequence Synthetic primer
gcactccagaggacagcrgtgtcaata

SEQ ID NO: 11 - DNA, 20 bp, Artificial Sequence
  Description of Artificial Sequence Synthetic primer
atttcctgtcccctgccttg

SEQ ID NO: 12 - DNA, 20 bp, Artificial Sequence
  Description of Artificial Sequence Synthetic primer
ttgctacccgtgatattgct

SEQ ID NO: 13 - DNA, 20 bp, Artificial Sequence
  Description of Artificial Sequence Synthetic primer
ggccagtctcagagccatac

(19) United States
(12) Patent Application Publication
(10) Pub. No.: US 2018/0031575 A1
(43) Pub. Date: Feb. 1, 2018

(54) Detecting Dementia and Alzheimer's Disease Associated Biomarkers Stabilized on Solid Support Materials

(71) Applicant: GE HEALTHCARE UK LIMITED, LITTLE CHALFONT (GB)

(72) Inventors: Peter James Tatnell, Wales (GB); Jeffrey Kenneth Horton, Wales (GB)

(21) Appl. No.: 15/525,715

(22) PCT Filed: Oct. 14, 2015
(86) PCT No.: PCT/US15/55554

§ 371 Date: May 10, 2017

Related U.S. Application Data
(60) Provisional application No. 62/082,199, filed on Nov. 20, 2014.

(51) Int. Cl. G01N 33/68; C12Q 1/68; C12Q 1/44

(52) U.S. Cl. CPC G01N 33/6896; G01N 33/6869; C12Q 1/44; C12Q 1/6883; G01N 2800/2814; G01N 2800/2821; G01N 2800/50; G01N 2800/52; G01N 2333/55; G01N 2333/922; C12Q 2600/158; C12Q 2600/118

(57) ABSTRACT

Embodiments of the invention provides a method for detecting one or more biomarkers derived from a body fluid, comprising performing one or more assays for the biomarkers from a sample of the body fluid, whereby the sample is previously preserved on a solid support; wherein a change in the biomarkers provides an indication of a biological event in the brain. The invention also provides related methods for identifying a person as being at risk of dementia or Alzheimer's disease, monitoring a person for the onset or progression of dementia or Alzheimer's disease, evaluating the effectiveness of a potential pharmaceutical agent.

### FIELD OF THE INVENTION

The present invention relates to methods for detecting dementia and Alzheimer's disease associated biomarkers stabilized on solid support materials.

### BACKGROUND OF THE INVENTION

Dementia currently affects 44 million individuals globally. This figure will rise to 135 million by 2050. The current global cost is $600 bn. Research into treatments for Alzheimer's disease has been plagued by failure. Between 2002 and 2012, over 99% of trials aimed at preventing or reversing the disease failed. All failures were attributed to treating patients when it is already too late, since symptoms appear around a decade after the start of the disease. Therefore identifying patients earlier is one of the priorities for dementia research.

Recently, researchers identified a set of plasma proteins in the blood which can predict the start of dementia and it has been proposed that these biomarkers may be used to identify individuals for developing blood test/trials for new dementia drugs. Proteins in the blood of 452 healthy people were compared to those from 220 with mild cognitive impairment and 476 with Alzheimer's disease. From this, researchers were able to tell with 87% accuracy which patients with mild cognitive impairment would go on to develop Alzheimer's disease. This result will allow the early identification of candidates, thereby increasing the chances of success for future clinical trials. These biomarkers also represent a potential means to identify people who will eventually progress to Alzheimer's disease and thus people who can be entered into clinical trials earlier. Early treatment will increase the potential of a positive drug effect and thereby therapy.

There is a need for improved sample storage and detection to facilitate the identification of people at risk of dementia or developing Alzheimer's disease.

### BRIEF SUMMARY OF THE INVENTION

This invention describes a novel method that facilitates biomarker detection and quantification which supports the detection of biomarkers associated with dementia and Alzheimer's disease. Blood, plasma or other relevant biological sample types are collected on a solid support, biomarkers such as proteins, RNA and DNA are stabilized and detected. These biomarkers remain stable on the solid support, and may be detected after a prolonged storage.

In one aspect, it is provided a method for detecting one or more biomarkers derived from a body fluid, comprising performing one or more assays for the biomarkers from a sample of the body fluid, whereby the sample is previously preserved on a solid support; wherein a change in the biomarkers provides an indication of a biological event in the brain.

In another aspect, it is provided a method for identifying a person as being at risk of dementia or Alzheimer's disease, comprising: detecting one or more biomarkers using a method according to certain aspects of the invention; and predicting whether the person is at risk of dementia or Alzheimer's disease based on the detection result.

In another aspect, it is provided a method for monitoring a person for the onset or progression of dementia or Alzheimer's disease, comprising: obtaining and preserving, over time, a number of biological samples from the person on solid supports; detecting one or more biomarkers using a method according to certain aspects of the invention from some of the preserved samples; and predicting the onset or progression of dementia or Alzheimer's disease based on change in detected biomarker over time.

In another aspect, it is provided a method for evaluating the effectiveness of a potential pharmaceutical agent, comprising: obtaining and preserving on solid supports, over time, a number of biological samples from a person having dementia or Alzheimer's disease, while the person is been treated using the potential pharmaceutical agent; detecting one or more biomarkers using a method according to certain aspects of the invention from some of the preserved samples; and predicting whether the agent is effective for treating the person having dementia or Alzheimer's disease.

Further details and advantages of the present invention will appear from the description and claims below.

### BRIEF DESCRIPTION OF THE DRAWINGS

FIG. 1 shows measurement of model protein (IL-2) from a solid support.

FIG. 2 shows measurement of model enzyme (DNase) from a solid support.

FIG. 3 shows measurement of model enzyme (RNase) from a solid support.

FIG. 4 shows result of direct amplification of a 500 bp genomic DNA fragment from human blood treated with heparin and preserved on various solid supports.

FIG. 5 shows result of direct PCR of 1 kb, 3.8 kb and 7.5 kb genomic DNA amplicons from human blood treated with EDTA and preserved on various solid supports.

FIG. 6 shows result of direct PCR performed on blood from several mammalian species treated with EDTA and preserved on 903 and FTA Gene Card sample collection cards.

FIG. 7 shows DNA amplification products derived from both the WT and NOS3 null gene knock-out mice.

FIG. 8 shows relative expression levels of GADPH from several tissue sources (blue/light and red/dark columns refer to different solid materials).

### DETAILED DESCRIPTION OF THE INVENTION

Various technologies provide opportunities for genomics and proteomics analysis; which can be used to evaluate altered expressions of gene and protein targets in blood or other tissue samples. The collection/storage of biomarkers for example in blood, and/or cerebral spinal fluid on solid supports can be followed by a simple direct or punch-in technique in which a sample on a solid support is added directly to detection reagents and subjected to biomarker detection methods such as, but not limited to, immunological assays and nucleic acid amplification/detection technologies without the prior purification of the biomarkers or analytes of interest.

The inventors have reviewed the importance of several genomic and proteomic biomarkers and/or other analytes of interest for their association with events in the brain, and suggest the collection of these markers on solid supports such as those supplied by Whatman/GE Healthcare.

Thus, in one embodiment, the invention provides a method for detecting one or more biomarkers derived from a body fluid, which method comprising performing one or more assays for the biomarkers from a sample of the body fluid, whereby the sample is previously preserved on a solid support; wherein a change in the biomarkers provides an indication of a biological event in the brain. In certain embodiments, the one or more assays include assays for a nucleic acid molecule, or protein based assays, or antibody based assays or enzyme based assays. In certain embodiments, the change may be an increase or decrease in the level of the biomarker. The change may also be the absence of a biomarker that is present in a control or normal individual, or vice versa. The change may also be a change at the genomic level (i.e., single nucleotide mutations, insertions, deletions or other mutations or polymorphism at the genomic level).

In certain embodiments, the body fluid is blood or cerebral spinal fluid.

A search of the scientific literature has identified about 25 dementia or Alzheimer's disease associated protein and nucleic acid biomarkers. While the expression of these biomarkers may be detected using protein, antibody, enzyme or RNA based gene expression assays, genomic mutations may be detected at the DNA level. A list of the biomarkers associated with dementia and Alzheimer's disease is described in table 1 below.

TABLE 1
Biomarkers associated with certain biological events in the brain

| Transthyretin | TTR | Hye et al 2014 |
|---|---|---|
| | | Alzheimers & |
| | | Dementia 1-9 |
| Clusterin | CLU | As above |
| Cystatin C | CysC | As above |
| A1AcidG | | As above |
| Intracellular adhesion molecule 1 | ICAM1 | As above |
| Cytochrome C4 | CC4 | As above |
| Pigment epithelium-derived factor | PEDF | As above |
| Alpha 1 antitrypsin | A1AT | As above |
| RANTES | CCL5 | As above |
| Apolipoprotein C3 | ApoC3 | As above |
| Apolipoprotein E (genotype) | ApoE | As above |
| Apolipoprotein A1 | ApoA1 | As above |
| Neuron-specific enolase | NSE | As above |
| Brain-derived neurotrophic factor | BDNF | As above |
| Complement factor H | CFH | Hye et al 2006 |
| | | Brain 129, |
| | | 3042-3050. |
| Alpha macroglobulin | A2M | As above |
| Serum Amyloid P | SAP | As above |
| Ceruloplasmin | CERP | As above |
| Amyloid beta | APP | Richens et al |
| | | 2014 Int J Mol |
| | | Epidemiol Genet 5, |
| | | 53-70 |
| Fibrinogen alpha chain precursor | FGA | As above |
| Keratin type 1 cytoskeletal 9 | KRT9 | As above |
| Serum albumin precursor | | As above |
| SPARC-like 1 protein | SPARC-like 1 | As above |
| plasminogen binding protein | Tetranectin | As above |
| Tau | | Wang et al 2007 |
| | | Eur J Neurosci 25, |
| | | 59-68 |
| Tau kinases | | As above |
| Tau phosphatases | | As above |
| Synuclein | | Irwin et al (2013) |
| | | Nature Reviews |
| | | Neuroscience 14; |
| | | 626-636 |

Assay methods for detecting protein and nucleic acid biomarkers are known. Such assays may be selected from the group consisting of: gene expression or protein expression profiling using RT-PCR, polymerase chain reaction (PCR), quantitative PCR (qPCR), isothermal amplification, immunological-PCR, microarray assays, enzyme linked immunosorbent assay (ELISA), immunological techniques, gel electrophoresis (2DE), capillary electrophoresis (TOF MS), high performance liquid chromatography (HPLC), mass spectrometry (MS), flame photometry, atomic absorption spectrophotometry, and visible spectrophotometry.

In certain embodiments, the sample is previously preserved on a solid support. The biological sample may simply be applied to the solid support and allowed to dry at ambient temperature for preservation.

In certain embodiments, the solid support is fibrous, for example a cellulose fibre material, or a glass fibre/microfibre material.

In certain embodiments, the solid support is a porous polymer, for example porous membrane material such as polyester, polyether sulfone (PES), polyamide (Nylon), polypropylene, polytetrafluoroethylene (PTFE), polycarbonate, cellulose nitrate, cellulose acetate, alginate or aluminium oxide.

In certain other embodiments, the support surface is impregnated with chemicals, the chemicals including: a weak base; a chelating agent; an anionic surfactant; and/or a chaotropic agent such as guanidinium thiocyanate.

In certain other embodiments, the solid support is, but not limited to, FTA paper, FTA Elute paper, Whatman 903 paper, or alginate coated support.

Herein FTA (including FTA microcards, FTA indicating, and FTA classic) is a cellulose fibre paper treated with stabilizing chemicals, for example a weak base, a chelating agent and an anionic surfactant, whereby the support surface is impregnated with the stabilization chemicals. In this way the biological sample materials can be stored as a dried material on the solid support for many months or even years, thereby allowing time for transportation of the solid support, if needed, to a laboratory, at an ambient temperature. Simple recovery is then possible, by for example purifying the biological sample materials from the solid support. Alternatively, the sample can be processed using direct or washed punch-in protocols. Storing a sample on the solid support also enables retesting the sample over time, by removing a portion of the sample and testing that portion as needed.

FTA Elute herein describes similar paper but coated with a chaotropic agent such as guanidinium thiocyanate. Herein Whatman 903 describes uncoated cellulose fibre paper.

Certain solid supports are described in WO 2012113911, WO 2012113907, WO 2012113906 and WO 2013165870, the disclosure of each is incorporated by reference in its entirety.

The solid supports described above are intended to be used in a generally flat configuration, but in the alternative, may for example be used on a roll.

In certain embodiments, the one or more assays is performed directly from a punch excised from solid support containing the sample. Thus, the assays may be carried out directly from punches excised from solid support on which a biological sample (i.e., blood) has been applied. The punches containing the sample may be added directly to an assay reaction. Optionally, simple “punch-ins” additions can be performed in which the excised punch (solid support plus sample) is washed to remove any potential inhibitory chemical prior to the addition to the reaction.

In certain embodiments, the biomarkers are isolated and/or purified from the solid support prior to detection. Methods for purifying protein and nucleic acid molecules from a sample dried on a solid support are known.

In some embodiments, more than one biomarker may be assayed. For example, 2, 3, 4 or 5 of the biomarkers may be detected to assess the biological event of interest in the brain. In some embodiments, all the biomarkers in Table 1 may be detected to assess the biological event of interest. The biomarkers may be detected using different assays, including those methods described above for the detection of protein and nucleic acid biomarkers.

In certain embodiments, the assays may be multiplexed. Thus, more than one biomarker may be detected in a single reaction.

In certain embodiments, the one or more assays is performed using lyophilized reagents. Lyophilized reagents such as GE Healthcare's illustra Ready-To-Go (RTG) products are well known. These reagents may contain primers to analyse specific nucleic acids e.g. RNA, DNA etc or antibodies to detect the specific protein biomarkers of interest.

In certain embodiments, the one or more assays is performed in the presence of cyclodextrin. Cyclodextrin acts as a sequestor of detergents which coat the outside of certain solid support, thus improved DNA amplification assays maybe performed including direct amplification assays.

In certain embodiments, the biomarkers are quantified after detection.

In certain embodiments, the sample is previously preserved on a solid support and stored at room temperature. Biological sample preserved on a solid support may be stable for a long period of time, see for example, GE Healthcare Life Sciences Application Note 29-0082-33 AA.

In certain aspects, it is provided a method for identifying a person as being at risk of dementia or Alzheimer's disease, comprising: detecting one or more biomarkers by a method according to certain embodiments of the invention; and predicting whether the person is at risk of dementia or Alzheimer's disease based on the detection result. In certain embodiments, the detected results are compared to controls from people with or without the risk of dementia or Alzheimer's disease.

In certain aspects, it is provided a method for monitoring a person for the onset or progression of dementia or Alzheimer's disease. The method comprises obtaining and preserving, over time, a number of biological samples from the person on solid supports; detecting one or more biomarkers according to certain embodiments of the invention from some of the preserved samples; and predicting the onset or progression of dementia or Alzheimer's disease based on change in detected biomarker over time.

In certain embodiments, the detecting step is performed using a portion of some of the preserved samples. In certain embodiments, the detecting step is repeated over time using portions of the preserved samples.

In certain embodiments, the method for monitoring a person for the onset or progression of dementia or Alzheimer's disease further comprises comparing the detected biomarker results to controls from people with or without the risk of dementia or Alzheimer's disease.

In certain aspects, it is provided a method for evaluating the effectiveness of a potential pharmaceutical agent. The method comprises obtaining and preserving on solid supports, over time, a number of biological samples from a person having dementia or Alzheimer's disease, while the person is been treated using the potential pharmaceutical agent; detecting one or more biomarkers according to certain embodiments of the invention from some of the preserved samples; and predicting whether the agent is effective for treating the person having dementia or Alzheimer's disease.

In certain embodiments, the detecting step is performed using a portion of some of the preserved samples. In certain embodiments, the detecting step is repeated over time using portions of the preserved samples.

In certain embodiments, the method for evaluating the effectiveness of a potential pharmaceutical agent further comprises comparing the detected results to controls from people with or without dementia or Alzheimer's disease.

### EXAMPLES

The following examples are intended only to illustrate methods and embodiments in accordance with the invention, and as such should not be construed as imposing limitations upon the claims.

### Example 1

### Direct Measurement of Interleukin from a Solid Support

Recombinant IL-2± carrier (R & D Systems; Cat. 202-IL-CF-10 μg; lot AE4309112 and Cat. 202-IL-10 μg; lot AE4309081 respectively) was dissolved in blood (TCS Biosciences) at 50 pg or 100 pg/μl. Aliquots (1 μl containing, 50 (B) or 100 (A) pg of IL-2) were applied to GE Healthcare 903 filter papers.

These samples were allowed to dry overnight at ambient temperature and humidity. 3 mm diameter punched disks were extracted from each paper type using the appropriately sized punch. Single discs were directly analysed for IL-2 with reagents from a fully configured IL-2 Quantikine ELISA kit (R & D Systems, Cat. D2050, lot 273275). Direct assays were carried out “punch in well”, i.e., where a portion of the 903 filter paper was punched out and deposited in a reaction well of a convention multiwall plate.

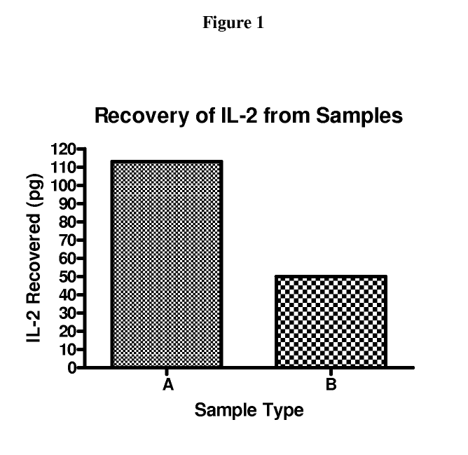

On completion of the assay the optical density was monitored at 450 nm. The recovery of IL-2 was determined by comparing values to a standard curve of known IL-2 concentrations. Recovery rates are shown in FIG. 1, and demonstrate that effective amounts of a protein can be recovered when the protein is deposited on a solid support.

TABLE 2
ELISA-based kits currently available

| Human Protein biomarker | Catalogue no | Detection Range | | Supplier |
|---|---|---|---|---|
| Transthyretin | SEA726Hu | 0.781-50 | ng/mL | Uscn Life Science Inc |
| Clusterin | SEB180Hu | 0.781-50 | ug/mL | Uscn Life Science Inc |
| Cystatin C | SCA896Hu | 88% | spiked serum | Uscn Life Science Inc |
| Intracellular adhesion molecule 1 | SCA548Hu | 78.13-5000 | pg/mL | Uscn Life Science Inc |
| Cytochrome C4 | | | | |
| PEDF | SCB972Hu | 0.16-120 | ng/mL | Uscn Life Science Inc |
| Alpha 1 antitrypsin | SCB697Hu | 80% | spiked serum | Uscn Life Science Inc |
| RANTES | SCA116Hu | 99% | spiked serum | Uscn Life Science Inc |
| Apolipoprotein C3 | SEB890Hu | 0.313-20 | ng/mL | Uscn Life Science Inc |
| Apolipoprotein A1 | SCA519Hu | 100% | spiked serum | Uscn Life Science Inc |
| Neuron-specific enolase | SEA537Hu | 0.625-40 | ng/mL | Uscn Life Science Inc |
| Brain-derived neurotrophic factor | SCA011Hu | 87% | spiked serum | Uscn Life Science Inc |
| Complement factor H | SEA635Hu | 23.44-1500 | ng/mL | Uscn Life Science Inc |
| Alpha macroglobulin | SCB017Hu | 87% | spiked serum | Uscn Life Science Inc |
| Serum Amyloid P | SCB539Hu | 0.55-400 | ng/mL | Uscn Life Science Inc |
| Ceruloplasmin | SCA909Hu | 1.37-1000 | pg/mL | Uscn Life Science Inc |
| SPARC-like 1 protein | SEM267Hu | 0.313-20 | ng/mL | Uscn Life Science Inc |
| plasminogen binding protein | Ab116694 | 219 | ng/ml | Abcam |

Many of the biomarkers in Table 1 may be detected using a commercially available ELISA kit, as illustrated in Table 2.

Thus, a protein from a biological sample such as blood or cerebral spinal fluid is stable on a solid support and may be detected and quantified using existing protein detection methods.

### Example 2

### Model Enzyme Detection from Cells or Enzymes Transferred to Solid Supports

Protein and enzyme testing was carried out with fully configured DNase and RNase Contamination Kits (DNase & RNase Alert QC Systems, catalogue codes AM1970 & AM1966, Life Technologies) according to the manufacturer's instructions.

A. Dideoxyribonuclease (DNase)

In a first series of experiments, 0.125-0.5 U of DNase was applied to FTA and 903 papers in 10 μl volumes. DNAse and RNase activity was measured as outlined below (Data not shown).

In a second series of experiments, 1.2 mm punches were taken from 10⁶ human embryonic stem cells (GE Healthcare; cell line ref: WCB307 GEHC 28) which had been applied to FTA and 903 papers in 10 μl volumes as above. DNAse and RNase activity was measured as outlined below.

In a third series of experiments, 1.2 mm punches were taken from 10⁶ human embryonic stem cells (GE Healthcare; cell line ref: WCB307 GEHC 28) containing either 0.5 U of DNase or 10 μU of RNase added to these cells which had been applied to FTA and 903 papers in 10 μl volumes.

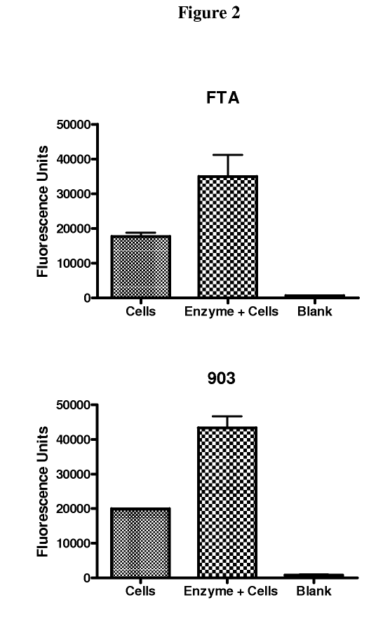

Detection of DNase activity was carried out as follows using a cleavable fluorescent-labelled DNase substrate. Each punch was ejected into separate wells of 96-well plates. Lyophilized DNase Alert Substrate was dissolved in TE buffer (1 ml) and dispensed (10 μl) into the test wells of the 96-well plate. 10× DNase Alert Buffer (10 μl) and nuclease-free water (80 μl) was added and the test solution (100 μl) incubated for 60 minutes at 37° C. The DNase Alert QC System Substrate is a modified DNA oligonucleotide that emits a pink fluorescence when cleaved by DNase. For this assay, fluorescence was measured on a Tecan Ultra (excitation/emission 535/595 nm using medium gain). Solutions containing DNase activity produced a pink fluorescence, whereas solutions without DNase activity did not fluoresce. Thus, higher levels of DNase corresponded to an increase in the amount of light output. Negative controls consisted of nuclease-free water (80 μl) in place of sample. DNAase activity can be detected and quantified in a rate dependent manner using the 903 or FTA papers as solid supports. FIG. 2 demonstrates that recovery of DNase and enzymatic activity is achieved on a FTA chemically treated filter paper (FTA) and a 903 untreated filter paper (903).

B. Ribonuclease (RNase)

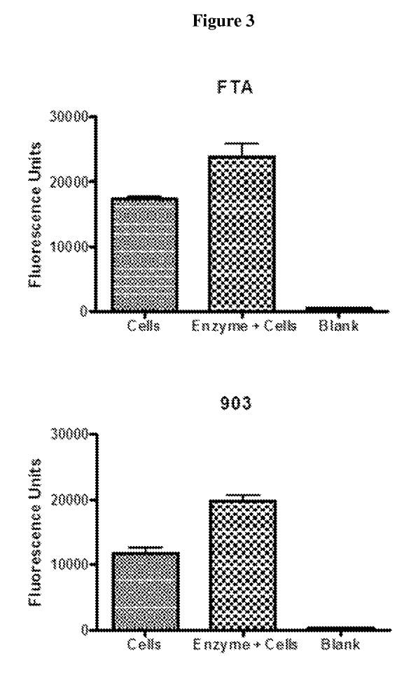

Detection of RNase was carried out as follows using a cleavable fluorescent-labelled RNase substrate. Each punch was ejected into separate wells of 96-well plates. Lyophilized RNase Alert Substrate was dissolved in TE buffer (1 ml) and dispensed (10 μl) into the test wells of the 96-well plate. 10× RNase Alert Buffer (10 μl) and nuclease-free water (80 μl) was added and the test solution (100 μl) incubated for 60 minutes at 37° C. The RNase Alert QC System Substrate is a modified RNA oligonucleotide that emits a green fluorescence when cleaved by RNase. For this assay, fluorescence was measured on a Tecan Ultra (excitation/emission 485/535 nm using medium gain). Solutions containing RNase produced a green fluorescence, whereas solutions without RNase activity did not fluoresce. Thus, higher levels of RNase corresponded to an increase in the amount of light output. Negative controls consisted of nuclease-free water (80 μl) in place of sample. RNAase activity can be detected and quantified in a rate dependent manner using the 903 or FTA papers (FIG. 3).

Thus, enzymes from a biological sample may be dried on a solid support and remain stable. Detection of enzyme activity and quantification of the enzymes may be performed using existing methods.

### Example 3

### Direct PCR from Blood Preserved on Whatman FTA and 903 Cards

Thermo Scientific Phusion Blood Direct PCR Kit was demonstrated to support the amplification of DNA directly from blood samples stored on a range of solid supports including Whatman 903, FTA and FTA Elute cards (Chum and Andre 2013; Thermo Fisher Scientific). FTA and FTA elute cards are examples of chemical coated paper-based cards whilst 903 cards are not chemically coated. In direct amplification workflows, no prior DNA extraction or purification steps are needed and the cards are simply added to the PCR reaction mixture.

Sample preparation: Fresh blood or blood preserved with heparin (1.4 IU/mL), K₂EDTA (1.8 mg/mL), or Na Citrate (109 mM) was applied to Whatman 903 Cards, FTA Elute Cards, or FTA Gene Cards and dried as per the manufacturer's instructions. For direct PCR, a 1 mm diameter disc was punched out of the sample in the card and used in the following PCR reaction volumes: Whatman 903: 10-50 μl, FTA Elute Card: 25-50 μl and FTA Gene Card: 50 μl.

When larger punches or smaller reaction volumes were used, punches were washed with 20 μL of water at 50° C. for 3 minutes. After removing the water, PCR components were added directly to the rinsed punch. The parameters and reagents used are listed in Tables 3, 4 and 5, below.

TABLE 3
PCR reaction mixtures

| | | | | | Final |
|---|---|---|---|---|---|
| Component | 25 μL Reaction | | 50 μL Reaction | | Conc. |
| H₂0 | Add to 25 | μL | Add to 50 | μL | |
| 2× Phusion | 12.5 | μL | 25 | μL | 1x |
| Blood PCR | | | | | |
| Buffer | | | | | |
| Primer F | x | μL | x | μL | 0.5 μM |
| (Forward) | | | | | |
| Primer R | x | μL | x | μL | 0.5 μM |
| (Reverse) | | | | | |
| Phusion | 0.5 | μL | 1 | μL | |
| Blood DNA | | | | | |
| Polymerase | | | | | |
| 903/FTA Card | 1 | mm punch | 1 | mm punch | |

Optional Components for Reaction Optimization*

| | | | | | |
|---|---|---|---|---|---|
| 50 mM MgCl₂ | 0.75 | μL | 1.5 | μL | |
| 50 mM EDTA | 0.8-1.25 | μL | 1.25-2.5 | μL | |
| DMSO | 1.25 | μL | 2.5 | μL | 5% |

TABLE 4
PCR thermo-cycling protocols. The 2-step protocol was used when
primer Tm values were 69-72° C.

| | 2-step Protocol | | 3-step Protocol | | |
|---|---|---|---|---|---|
| Cycle Step | Temp. | Time | Temp. | Time | Cycles |
| Lysis of cells | 98° C. | 5 minute | 98° C. | 5 minute | 1 |
| Denaturation | 98° C. | 1 s | 98° C. | 1 s | 35-40 |
| Annealing* | — | — | x° C. | 5 s | |
| Extension** | 72° C. | 15-30 s/ | 72° C. | 15-30 s/ | |
| | | kb | | kb | |
| Final extension | 72° C. | 1 minute | 72° C. | 1 minute | 1 |
| | 4° C. | hold | 4° C. | hold | |

TABLE 5
Primers used to amplify the exemplary genes of interest

| | Amplicon | | Annealing |
|---|---|---|---|
| | Length | Forward Primer | Temperature |
| Gene of Interest | (kb) | Reverse Primer | (° C.) |
| Cathepsin K gene | 0.5 | GAGAATCGCTTGAACCCGGGAGGTGTAGGT | 78.1 |
| | | CCTGCTGATGCCTGGCCTCTTTCTTCTTTG | 78.1 |
| Glutathione | 1.0 | CATCAGCCCGTCTAGGAACCCAGTCATCAG | 77.6 |
| peroxidase 3 | | CTCCTTCATCCCGCTACACCACGCATACAC | 77.9 |
| Beta-globin gene | 3.8 | GCACTGGCTTAGGAGTTGGACT | 65.9 |
| | | ACAGACACCCAGGCCTACTTG | 65.6 |
| Beta-globin gene | 7.5 | GCACTGGCTTAGGAGTTGGACTTCAAACC | 73.9 |
| | | CAACTGCTGAAAGAGATGCGGTGGG | 75.1 |
| SOX21 gene 5′ region | 0.2 | AGCCCTTGGGGASTTGAATTGCTG | 73.5 |
| (Control Primers | | GCACTCCAGAGGACAGCRGTGTCAATA | 72.2/75.3 |
| of Phusion Blood | | | (R = A/G) |
| Direct PCR Kit) | | | |

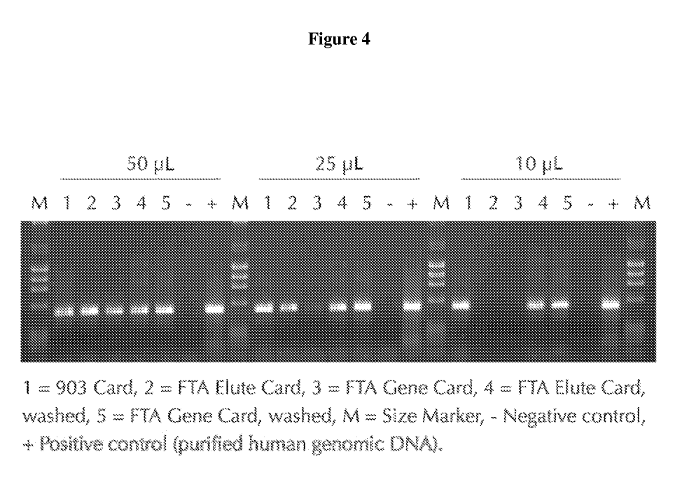

FIG. 4 shows result of direct amplification of a 500 bp genomic DNA fragment from human blood treated with heparin and preserved on various cards. Reactions were performed from 1 mm punches either rinsed or placed directly into PCR reactions of 50, 25 or 10 μl in volume. A 2-step PCR protocol described in Materials and Methods was used.

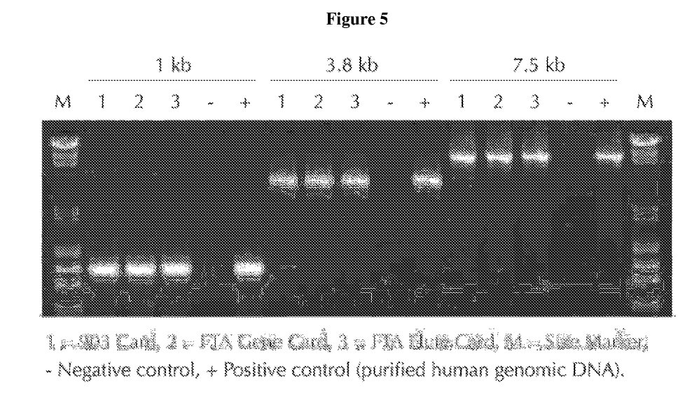

FIG. 5 shows result of direct PCR of 1 kb, 3.8 kb and 7.5 kb gDNA amplicons from human blood treated with EDTA and preserved on various cards. Reactions were performed from 1 mm punches in 50 μl reactions (FTA Gene Card punches were washed by rinsing with water for 7.5 kb fragment). A 2-step protocol was used for 1 kb and 7.5 kb fragments and a 3-step protocol for 3.8 kb amplicon.

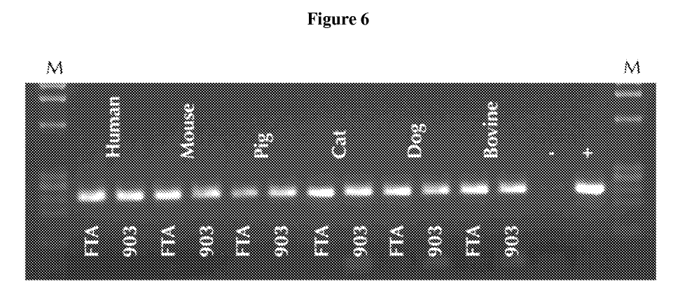

FIG. 6 shows result of direct PCR performed on blood from several mammalian species treated with EDTA and preserved on 903 and FTA Gene Cards. Reactions were performed from 1 mm punches using the universal control primers included in the Phusion Blood Direct PCR Kit and 20 μl reaction volume (FTA Gene Cards were rinsed). M Size Marker, − Negative control, + Positive control (purified human genomic DNA).

The PCR study confirmed that DNA can be directly amplified from blood stored on various filter cards.

Samples derived from the 903 Cards showed almost no inhibition, and a 1 mm punch could be used with reaction volumes as low as 10 μl. FTA Elute and FTA Cards exhibited varying levels of inhibition. FTA elute inhibited direct PCR reactions slightly; a 1 mm disc in a 25-50 μl reaction worked well, but when placed in a 10 μl reaction, the PCR was totally inhibited. FTA Gene Cards showed the greatest level of inhibition. Without any pre-treatments, a 1 mm punch of FTA Gene Card worked well only in a 50 μl reaction volume. For smaller reaction volumes, a very simple washing protocol was enough to remove inhibitors from both FTA Elute and FTA Gene Cards. After washing the card punch for 3 minutes with water, the sample was of sufficient purity for use in direct PCR reactions with Phusion Blood Direct PCR Kit at all reaction volumes tested.

Punches from 903 Cards and rinsed punches from FTA Elute and FTA Gene Cards (all 1 mm in diameter) were used in 50 μl reaction volumes with primers specific for 1 kb, 3.8 kb and 7.5 kb amplicons. In all cases, the PCR reaction generated the appropriately sized amplification product.

The Phusion Blood Direct PCR Kit is compatible with blood from variety of species. A highly conserved 237 bp region upstream of the SOX21 gene (A. Woolfe, M. Goodson, PLoS Biol. 3, e7; 2004) was successfully amplified from blood of a number of vertebrate species dried onto 903 and FTA Gene Cards.

### Example 4

### Genotyping Using Biological Samples Applied to Solid Support Materials

Many cancers are associated with genetic rearrangements. The Ewing sarcoma breakpoint region 1 (EWSR1) is translocated in many sarcomas. Recently, its rearrangement has been described in salivary gland hyalinizing clear cell carcinomas (Shah AA et al 2013 Am J Surg Pathol. 37:571-8 EWSR1 genetic rearrangements in salivary gland tumors: a specific and very common feature of hyalinizing clear cell carcinoma). The study illustrates the potential of solid support material and the idea described in this document to potentially screen for such genetic rearrangements within a complex mammalian genome.

### DNA Sample Collection, Storage and Detection

Murine tissues from c57BL/6 mice and NOS3 null mice (in a 129/B6 background) were applied to a range of different paper-based solid supports. The mice were euthanized and dissected to collect organs (blood, heart, brain, lung, liver, and kidney). The Organs were ‘sandwiched’ between two paper layers. Pressure was applied via a sterile pipette to imbed tissues in each of the cellulose matrices. For tissue homogenate, approximately 5 mg of tissue was processed using a plastic dounce homogenizer in a 1.5 ml microfuge tube and then subsequently applied to the appropriate paper matrix. After application all the samples were allowed to air-dry for 2 hours prior to storage in a sealed pouch with desiccant. In some instances samples were stored up to 2 months before processing.

### DNA Genotyping, and Quantitation

A Harris disposable micro punch (1.2 mm or 3 mm diameter) was used to excise the dried tissue samples from the paper cards respectively in the form of punched disks. The sample disk was excised from the center of the dried sample and placed in a clean DNase free-1.5 ml micro-centrifuge tube.

Null or gene knockout NOS3 mice were identified by PCR amplification of genomic DNA with endothelial Nitric Oxide Synthases (eNOS) exon 10-specific forward primer (5′-ATT TCC TGT CCC CTG CCT TG-3′), eNOS Neo-specific forward primer (5′-TTG CTA CCC GTG ATA TTG CT-3′), and eNOS exon 12-specific reverse primer (5′-GGC CAG TCT CAG AGC CAT AC-3′).

Target DNA's were amplified with an initial 10 min denaturation step followed by 36 cycles of 94° C. for 35 sec, 65° C. for 1 min, and 72° C. for 1 min; followed by a final extension at 72° C. for 5 min. using a MJ Research thermo-cycler. The resultant PCR products were visualized with using an Experion capillary electrophoresis system. Mouse DNA quantification was achieved using the Primer Design genomic DNA quantification kit for mouse samples (gDNA-mo-q-DD) following manufacturer's instructions. Individual wild type (WT) and NOS3 null tissue samples were applied separately to different paper cards. In order to exemplify the ability to differentiate genotypic variants from DNA stored on the paper matrices, PCR amplification of a region was carried out on WT and transgenic (NOS3 null, gene knock-out) mice.

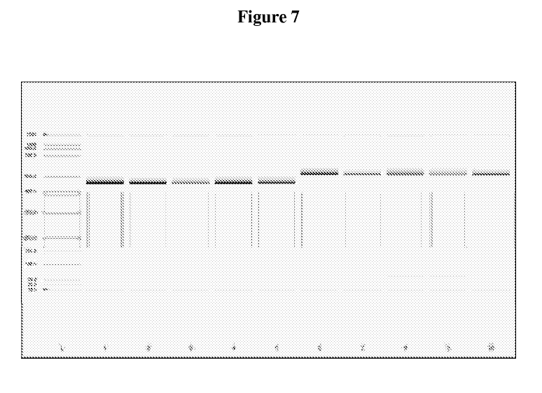

In FIG. 7 PCR amplicons are shown, associated with the NOS locus using DNA as an amplification template isolated from tissues from the paper cards. Lanes 1-5 are DNA isolated from WT mouse tissue (Heart, Liver, Brain, Lung, and Kidney respectively). Lanes 6-10 are DNA amplified from NOS mouse tissues (Heart, Liver, Brain, Lung, and Kidney respectively).

TABLE 6
Summary of the amplification of DNA isolated from tissues stored
on various solid support materials.

| | DNA | Support | Support | Support | Support |
|---|---|---|---|---|---|
| DNA type | Source | Material A | Material B | Material C | Material D |
| Wild Type | Blood | + | ND | ND | ND |
| Tissue DNA | Heart | ND | + | + | + |
| | Liver | ND | + | + | + |
| | Brain | ND | + | + | + |
| | Lung | ND | + | + | + |
| | Kidney | ND | + | + | + |
| Knock Out | Blood | + | ND | ND | ND |
| Tissue DNA | Heart | ND | + | + | + |
| | Liver | ND | + | + | + |
| | Brain | ND | + | + | + |
| | Lung | ND | + | + | + |
| | Kidney | ND | + | + | + |

(ND = not determined)

In Table 6 the successful amplification of DNA isolated from tissues stored on various solid support materials is recorded. DNA was isolated from a 1.2 mm punch. ‘+’ signifies the presence of amplicons.

FIG. 7 and Table 6 (above) show the DNA amplification products derived from both the WT and NOS3 null gene knock-out mice respectively. Results indicate that for both sample sources, the correctly sized DNA amplicons were produced from DNA isolated from all organ/tissue sources applied to the solid paper-support matrix. These data indicate that 1.2 mm Harris micro-punches can excise sufficient DNA from tissue stored on the solid paper supports to differentiate two genetic variants.

### Example 5

### RNA Collection, Purification and Quantitation

Tissue samples were applied to solid support paper cards as described. Sample punches were excised and the RNA isolated using the GE Healthcare illustra RNAspin kit as described below. RNA quantitation was performed on an ABI 7900 real time PCR system utilizing the commercially-available mRNA quantification kits.

Using a Harris 3 mm disposable micro punch, a punch was excised from the center of the dried sample spot and place in a clean RNase-free 1.5 ml micro-centrifuge tube. The illustra buffer RA1 (350 μl) was combined with 3.5 μl β-mercaptoethanol and the solution was added to the disc. The disc was homogenized using a 20 gauge needle. The resultant homogenate was transferred to the RNAspin Mini filter column for subsequent removal of residual material. The column was centrifuged for 1 min at 11,000×g. and the RNAspin Mini Filter discarded. The homogenized lysate contains the RNA and this filtrate was transferred to a new RNase-free 1.5 ml micro-centrifuge tube.

Ethanol (70%; 350 μl) was added to the homogenized lysate and mixed by vortexing for 2×5 sec pulses. For each preparation, the lysate was pipette up-and-down 2-3 times, and applied to an RNA Mini-spin column placed in a 2 ml micro-centrifuge tube. The tubes were centrifuged for 30 sec at 8000×g and the flow through discarded. The RNA spin column was transferred to a new collection tube.

The illustra MDB buffer (350 μl) was added and the tube centrifuged at 11 000×g for 1 min. Once again the flow-through was discarded and the column returned to the collection tube. A DNase reaction mixture was prepared according to manufacturer's instructions and was added to the surface of the filter contained within the RNAspin column. This DNAse incubation was performed at room temperature for 15 min.

The wash buffer RA2 (200 μl) was applied to the RNA Mini-spin column and the column was centrifuged for 1 min at 11 000×g. Once again the flow-through was discarded and the column returned to the collection tube.

Buffer RA3 600 μl was applied to the RNA Mini-spin column and the column centrifuged for 1 min at 11 000×g the flow-through was discarded and the column returned to the collection tube. An addition column wash with buffer RA3 (250 μl) was also performed. In order to dry the membrane completely, the column was centrifuged for 2 min at 11 000×g and the column finally placed into a nuclease-free 1.5 ml micro-centrifuge tube.

RNase-free water (40 μl) was applied to the column and the column centrifuged at 11 000×g for 1 min. The purified RNA was either used immediately in downstream applications or stored at −80° C. until used.

To determine the integrity of RNA from multiple tissues after prolonged storage, real-time reverse transcription polymerase chain reaction (RT-PCR) was carried out on RNA isolated from mouse tissue samples stored on the paper cards. These were stored in the presence of a desiccant for 2 months. mRNA quantification was accomplished according to manufacturer's instructions using either i) the ABI Taqman rodent GAPDH control kit (part #4308313), ii) the Invitrogen real-time LUX mRNA primer sets for murine HPRT, GAPDH, and Beta-Actin genes (cat. 105M-02, 100M-02, and 101M-02 respectively) or iii) tissue specific gene primer sets from Applied Bio-systems.

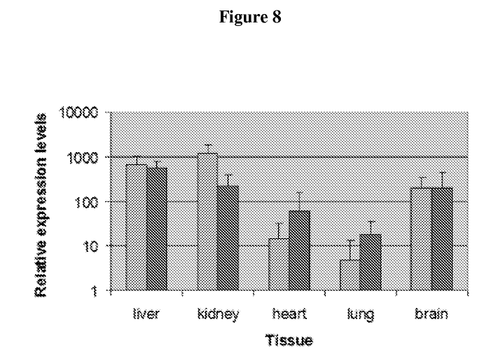

FIG. 8 shows the relative expression levels of GADPH from several tissue sources using the ABI Taqman rodent GAPDH control kit. RNA levels derived from samples applied to two different solid support cards were determined by comparison to known values generated from a quantification titration curve from mouse RNA standard samples. Comparable GAPDH RNA levels were detected from RNA isolated from both paper types.

Absolute quantitation of murine mRNA encoding HPRT, GAPDH and Beta-Actin was carried out with the appropriate Invitrogen real-time LUX primer sets. RNA levels derived from samples applied to the two different solid support cards were determined by comparison to known values generated from a quantification titration curve from mouse RNA standard samples. Data associated with the isolation of RNA is described in FIG. 8 and demonstrate that the support materials are able to support the storage and stabilization of RNA from numerous tissue types.

While the particular embodiment of the present invention has been shown and described, it will be obvious to those skilled in the art that changes and modifications may be made without departing from the teachings of the invention. The matter set forth in the foregoing description and accompanying drawings is offered by way of illustration only and not as a limitation. The actual scope of the invention is intended to be defined in the following claims when viewed in their proper perspective based on the prior art.

1. A method for detecting one or more biomarkers derived from a body fluid, comprising
performing one or more assays for said biomarkers from a sample of said body fluid, whereby the sample is previously preserved on a solid support;
wherein a change in said biomarkers provides an indication of a biological event in the brain.

2. The method of claim 1, wherein the body fluid is blood or cerebral spinal fluid.

3. The method of claim 1, wherein said biomarkers are one or more of Transthyretin, Clusterin, Cystatin C, A1AcidG, Intracellular adhesion molecule 1, Cytochrome C4, Pigment epithelium-derived factor, Alpha 1 antitrypsin, RANTES, Apolipoprotein C3, Apolipoprotein E (genotype), Apolipoprotein A1, Neuron-specific enolase, Brain-derived neurotrophic factor, Complement factor H, Alpha macroglobulin, Serum Amyloid P, Ceruloplasmin, Amyloid beta, Fibrinogen alpha chain precursor, Keratin type 1 cytoskeletal 9, Serum albumin precursor, SPARC-like 1 protein, plasminogen binding protein, tau, synuclein, tau kinases and tau phosphatases.

4. The method of claim 1, wherein the solid support is fibrous, a cellulose fibre material, or a glass fibre/microfibre material.

5. The method of claim 1, wherein the solid support is a porous polymer, a porous membrane material such as polyester, polyether sulfone (PES), polyamide (Nylon), polypropylene, polytetrafluoroethylene (PTFE), polycarbonate, cellulose nitrate, cellulose acetate, alginate or aluminium oxide.

6. The method of claim 1, wherein the support surface is impregnated with chemicals, said chemicals including: a weak base; a chelating agent; an anionic surfactant; and/or a chaotropic agent such as guanidinium thiocyanate.

7. The method of claim 1, wherein performing one or more assays is carried out directly from a punch excised from solid support containing the sample, the method optionally comprises a step of washing the excised punch to remove any potential inhibitory chemical prior to the assay reaction.

8. The method of claim 1, further comprising a step of purifying the biomarker from the solid support prior to detecting the biomarker.

9. The method of claim 1, wherein two or more of said biomarkers are assayed.

10. The method of claim 1, wherein the one or more assays are different assays selected from assays for a nucleic acid molecule, or protein based assays, or antibody based assays or enzyme based assays.

11. The method of claim 1, wherein the one or more assays are multiplexed.

12. The method of claim 1, further comprising quantifying at least one of the biomarkers.

13. The method of claim 1, wherein said sample is previously preserved on a solid support and stored at ambient temperature.

14. The method of claim 1, wherein said change is selected from an increase or decrease in the level of the biomarker, the absence of a biomarker that is present in a control or normal individual, the presence of a biomarker that is absent in a control or normal individual and DNA polymorphism or mutation at the genomic level.

15. The method of claim 1, wherein said one or more assays include assays for a nucleic acid molecule, or protein based assays, or antibody based assays or enzyme based assays.

16. A method for identifying a person as being at risk of dementia or Alzheimer's disease, comprising:
detecting one or more biomarkers according to the method of claim 1; and
predicting whether the person is at risk of dementia or Alzheimer's disease based on the detection result.

17. The method of claim 16, further comprising comparing the detected results to controls from people with or without the risk of dementia or Alzheimer's disease.

18. A method for monitoring a person for the onset or progression of dementia or Alzheimer's disease, comprising:
obtaining and preserving, over time, a number of biological samples from said person on solid supports;
detecting one or more biomarkers according to the method of claim 1 from some of the preserved samples; and
predicting the onset or progression of dementia or Alzheimer's disease based on change in detected biomarker over time.

19. The method of claim 18, wherein the detecting step is performed using a portion of some of the preserved samples.

20. The method of claim 19, wherein the detecting step is repeated over time using portions of the preserved samples.

21. The method of claim 18, further comprising comparing the detected biomarker results to controls from people with or without the risk of dementia or Alzheimer's disease.

22. A method for evaluating the effectiveness of a potential pharmaceutical agent, comprising:
obtaining and preserving on solid supports, over time, a number of biological samples from a person having dementia or Alzheimer's disease, while the person is been treated using the potential pharmaceutical agent;
detecting one or more biomarkers according to the method of claim 1 from some of the preserved samples; and
predicting whether the agent is effective for treating said person having dementia or Alzheimer's disease.

23. The method of claim 22, wherein the detecting step is performed using a portion of some of the preserved samples.

24. The method of claim 23, wherein the detecting step is repeated over time using portions of the preserved samples.

25. The method of claim 22, further comprising comparing the detected results to controls from people with or without dementia or Alzheimer's disease.
